(19) United States
(12) Patent Application Publication
(10) Pub. No.: US 2007/0122874 A1
(43) Pub. Date: May 31, 2007

(54) Novel microbial consortium and use thereof for liquefaction of solid organic matter

(76) Inventors: Rangasamy Suthanthararajan, Chennai (IN); Kalyanaraman Chitra, Chennai (IN); Balasubramanian Umamaheswari, Chennai (IN); Ethirajulu Ravindranath, Chennai (IN); Sengodagounder Rajamani, Chennai (IN)

Correspondence Address: SEED INTELLECTUAL PROPERTY LAW GROUP PLLC, 701 FIFTH AVE, SUITE 5400, SEATTLE, WA, 98104, US

(21) Appl. No.: 11/368,347

(22) Filed: Mar. 3, 2006

(30) Foreign Application Priority Data
Nov. 25, 2005 (IN) 3155/DEL/2005

(51) Int. Cl. C12N 1/20; C12P 1/00

(52) U.S. Cl. 435/41; 435/252.4; 210/601

(57) ABSTRACT

The present invention relates to a novel process for liquefaction of solid organic matter by biological method. By this process the solid organic matter can be hydrolyzed using anaerobically treated water containing a consortium of fermentative micro-organisms thus resulting in an organic liquid which can be digested easily when applied to an anaerobic reactor for the recovery of valuable product namely biogas. The consortium comprises of the fermentative microorganism selected from the bacteria of the genera Bacteroides, Clostridium, Lactobacillus, Streptococcus, Peptococcus, Selenomonas in any combination. This process has potential application in all the industries wherever the solid organic matters are generated as a waste posing a major problem for their disposal, adding thereby to environmental pollution.

### CROSS-REFERENCE TO RELATED APPLICATION

This application claims priority from Indian Patent Application No. 3155/DEL/2005, filed Nov. 25, 2005, now pending, which application is incorporated herein by reference in its entirety.

### BACKGROUND OF THE INVENTION

1. Field of the Invention

The present invention relates to a novel microbial consortium and use thereof for liquefaction of solid organic matter. More particularly, the present invention relates to novel process for liquefaction of solid organic matter by biological method. The process is envisaged to have enormous application in tanning industry for effective disposal of various solid wastes, which otherwise add to the pollution load. It can also be used for treating the solid byproducts generated in the slaughterhouses. Further, it is envisaged to have enormous application in treating the solid organic matter, generated as wastes in different consumer product industries, food processing industries, sago and starch industries. Moreover, the process has potential application for effectively disposing the agricultural wastes. It may also find application in treating the wastes generated from chicken broiler stalls, meat and fish stalls, poultry farms, vegetable and fruit markets, restaurants, hostels, residences and commercial complexes. The solid organic substances, which are generated as wastes in connection with all the above applications posing a major problem for their disposal, adding thereby to environmental pollution, are essentially converted into liquid by the process of the present invention, so that the said liquefied organic matter can further be treated either aerobically or anaerobically to result in carbon-di-oxide, water and biogas. The biogas, so generated, can primarily be used as fuel. It can also be used in large scale for generating electricity.

2. Description of the Related Art

It is well known that the disposal of organic materials is becoming increasingly difficult and expensive. Many industries like tanneries, municipal sewage treatment plant, slaughterhouse, poultry industry, biological effluent treatment units etc produce waste containing biodegradable organics. In India the average per capita solid waste generation is 500 gm per day. The estimated generation of wastes per day in class-1 cities is 30 million tons. The organic solid wastes generated from industries are 300,000 tons per day, as reported by Contractors FEC Consultants Ltd, Delphi International Ltd, (Evaluation of energy from waste investments opportunities in India, Chapter 5, Page No.18-20). Solid organic matter is generated from domestic refuse and various industries like Slaughter house, Sago and Starch industries, Tannery fleshing from buff and cow hides and skins, shaving, and trimming of tanned hides and skins from the leather industries, Fruit and vegetable market waste, Food processing wastes, Intestine that comes out from the chicken broiler stall, Wastes from meat and fish stall, Organic waste from cooked food remains generated from hostels, restaurants, office complexes and commercial areas.

The major solid wastes generated from leather industries are fleshing, shaving, trimming and buffing dust. Flesh is a thin layer appended to the corium. About 15% of the total thickness of raw skin is removed as flesh in beam house operation in tanneries. The remaining 85% of the skin is the actual leather making material. The flesh layer is removed from the skin/hide by a sharp fleshing knife or a fleshing machine. It is the process of removal of the subcutaneous fat and connective tissue from the flesh side of the skin. Fleshing operation can be carried out before or after the liming process. Fleshing mainly consists of protein, fat and lime. The fleshing generated in tanneries varies in size from 30-45 cm long and 2 to 10 cm in width. In India about 70,000 to 100,000 tons of wet fleshing with about 85% of moisture is generated per year as reported by E. Ravindranath et al: (Application of fleshing to Upflow Anaerobic Sludge Blanket (UASB) Reactor with tannery effluent for Biogas generation at CLRI, Chennai, India, July-99). At present about 10% of fleshing is used for manufacturing of glue and animal protein and remaining dumped as waste on land with other solid wastes as reported by UNIDO (Manual on Design, operation and Maintenance of tannery effluent treatment plants, Based on the papers presented during the UNIDO Regional workshop, Chennai, India. Unit 2: Cleaner technologies in Leather Production & Reduction of Pollution at Source, 13-24, Oct. 1997). This leads to leachate problem and pollution of ground water.

The poultry industry is a major food industry in India. About 1400 metric tones of solid wastes are generated from poultry in the country on an average per year (Technology Information, Forecasting and Assessment Council (TIFAC) /Techno Market Survey (TMS), Utilisation of slaughterhouse waste material for the preparation of animal feed, Pp217-222, 2002). Wastes generated from each bird are about 300 gm. Of this the intestinal waste is about 65-140 gm depending on the size of the bird. At present the intestinal wastes are dumped as waste in open land, sewers and running streams that will result in blockage in sewerage pipelines and cause flooding of the pipes that result in offensive odor.

Wastes generated from fruit and vegetable market are approximately 50,000 tons/day. It normally consists of discarded green leaves, sub standard vegetables and fruits, fibrous materials. In majority of places Vegetable and fruit market waste are dumped in open land, which leads to leaching and odor problem.

Aerobic digestion (composting) and anaerobic digestion (biomethanation) are tried for the disposal of market waste as reported by Contractors FEC Consultants Ltd, Delphi International Ltd. (Evaluation of energy from waste investments opportunities in India, Chapter 7, Page no. 38-42). The limitation of the aerobic process is that aerobic composting does not normally provide recoverable energy. For the anaerobic digestion the waste has to be shredded and then mixed with water to form slurry prior to anaerobic digestion. It requires higher Hydraulic Retention Time (HRT) of 25 days in the anaerobic reactor for biogas generation.

At present organic solid wastes are being dumped off in open land, which results in offensive odor, ground water contamination due to leachate and occupancy of large land area. The biodegradable organic solid waste is processed by anaerobic digestion with biogas recovery. The anaerobic digestion process involves the mineralization of organic material to methane and carbon-di-oxide and it is accomplished by the action of various metabolic groups of microorganisms. For anaerobic digestion, the organic solid wastes are cut into small pieces using cutter for vegetable waste and mincer for the intestinal wastes and tannery fleshing. The above process converts the solid organic matter only into small solid pieces but without any hydrolysis. These solid pieces are digested for biogas recovery in anaerobic solid digester. But the limitation of this process is that it needs higher Hydraulic Retention Time (HRT) of 25-40 days in the anaerobic reactor for biogas generation. More over scum formation due to floating of feed material and blocking of inlet pipes reduces the performance efficiency of the solid digester. Another limitation of this process is that it requires capital investment for electrical and mechanical appliances, and operation & maintenance cost.

Many researches are being done for the aerobic and anaerobic fermentation for the biodegradable organic wastes. Most of the works published on the aerobic fermentation are done in thermophilic range of about 50° C. to 80° C. as described in U.S. Pat. Nos. 3,462,275 and 4,292,328.

In U.S. Pat. No. 5,810,903 a process for conversion of a wide variety of organic waste to useful protein enriched products suitable for use as animal feed, fertilizer, soil conditioner etc has been described. In this process first the waste is macerated before forming an aqueous mixture. The mixture is oxygenated and heated to thermophilic temperature over a period of about 2 to 6 days.

Annapurna et al. (Enzymatic hydrolysis of Tannery fleshings using chicken intestine proteases, Animal feed science technology 66 (1997) 139-147) treated limed fleshings with hydrochloric acid for 4-6 hours to remove the lime. After deliming, fleshings were mixed with minced chicken intestine containing proteolytic enzyme. Fleshing and chicken intestine were mixed in the ratio of up to 4:1 on the basis of weight. Along with it, mixture of formic acid, acetic acid and sulfuric acid were also added in order to provide pickling effect and the optimum pH of 2.0-3.0 for the enzyme action. The rate of hydrolysis was measured by determining the amount of liberated tyrosine.

In U.S. Pat. No. 4,473,589 (protein liquefaction process and products) sources of protein, such as residues and waste products from processing fish, poultry, pork and beef as well as single cell microorganisms are hydrolyzed to provide liquid products containing substantially amino acids, lipids and phosphorus. This process involves a brief alkaline treatment with heat (120-170° F.) and pH above 12 which facilitates liquefaction and enhances susceptibility to subsequent enzyme hydrolysis with bacterial protease (alkaline protease 201D, neutral protease 200 or highly alkaline protease available in united states) at elevated temperatures (100-140° F.) for 2-48 hours. Cell rupture and protein denaturation occur during alkaline treatment and permit and facilitates the enzymes to rapidly breakdown the intact proteins to smaller more soluble molecules.

U.S. Pat. No. 6,013,183 (Methods of liquefying microorganisms derived from biological wastewater treatment processes) explains about the process of liquefying the microorganisms. The process includes the step of passing slurry of the sludge at a high pressure through a nozzle having a restricted flow area to cause liquefaction of the microorganisms as they discharged from the nozzle at exceeding 10000-20000 psi pressure. First sludge is treated with alkali to pH 11 followed by maceration. Then the slurry is discharged from the nozzle of 10000-20000 psi pressure to atmospheric pressure causes the lysis and liquefaction of the microorganisms in the sludge.

Esa A. Salminen et al., described the anaerobic digestion of poultry slaughterhouse waste (Semi continuous anaerobic digestion of solid poultry slaughterhouse waste: effect of hydraulic retention time and loading, Water Research 36 (2002) 3175-3182). Poultry slaughterhouse waste that contained minced and mixed fractions of bone and trimmings, blood, offal, feathers (autoclaved at 120° C. for 5 minutes) was used as the feed for the anaerobic digestion. The mixture was first minced before feeding into the anaerobic reactor for digestion; the minced mixture was diluted with distilled water before feeding into the anaerobic reactor for biogas generation. Feeding to the reactor was done in the fill and draw method. HRT of about 13 to 100 days was maintained in the reactor.

L. Masse et al. (Effect of hydrolysis pretreatment on fat degradation during anaerobic digestion of slaughter house wastewater, Process biochemistry 38, (2003),1365-1372) suggested that pretreatment to hydrolyze part of the fat particles could accelerate the anaerobic treatment of slaughter house wastewater, The objective of this study was thus evaluated the effect of an hydrolysis pretreatment with pancreatic Lipase (PL-250) on the anaerobic digestion at 25° C. of pork fat particles in slaughter house wastewater.

J. Rodriquez et al. (Solid state anaerobic digestion of unsorted municipal solid waste in a pilot plant scale digester, Bioresource technology, 63(1998) 29-35) carried out a pilot scale studies for the municipal solid waste by reproducing the behavior of the sanitary landfill site. Water was introduced from the top in order to simulate rainfall over the sanitary landfill. The leachate collected was neutralized with sodium hydroxide and sodium bicarbonate and recycled.

Reduction of the size of the fat particles in slaughterhouse wastewater were tested by L. Masse et al. (Testing of alkaline and enzymatic hydrolysis pretreatments for fat particles in slaughterhouse wastewater, Biosource Technology 77 (2001) 145-155) using sodium hydroxide, and three lipases of plant, bacterial and animal origin. This was done to improve the anaerobic digestion of unsettled slaughterhouse wastewater. 4 hour pretreatment were done using sodium hydroxide and different lipases to study the effect of pretreatment on anaerobic digestion.

A laboratory scale system consisting of a solid bed batch reactor and an upflow packed bed methane phase reactor was installed by E. R. Vietez et al. (Biogasification of solid wastes by two phase anaerobic fermentation Biomass and bioenergy 16 (1999) 299-309). Both the reactors were inoculated with anaerobic digester effluent obtained from a municipal wastewater treatment plant. The solid bed reactor was packed with 2 cm chopped municipal solid waste. About 38% of the solid bed was hydrolyzed in 60 days with intermittent recirculation of the leachate. Performance of the solid bed was affected due to end product inhibition. This condition was improved by conveying the methanogenic effluent to the solid bed.

For the treatment of these solid wastes, various treatment methods like incineration, composting, aerobic and anaerobic digestion are available. Maceration of sludge is done before thermal conditioning. For incineration, energy input is needed for burning the solid wastes. Composting by aerobic and anaerobic method require long HRT. Most of the aerobic digesters are thermophilic. The temperature of the reactors has to be maintained to about 50° C. −80° C. For the digestion continuous supply of oxygen is needed. For the anaerobic digestion of solid wastes like municipal solid waste, poultry waste, slaughterhouse waste size reduction of the particle is done as a pretreatment before feeding into the reactor for the easy accessibility of the microorganisms to act on the substrate. External addition of enzymes is done to increase the rate of digestion. Chemicals were used to neutralize the leachate before recirculating to the reactor. Addition of lipases or sodium hydroxide is done to reduce the size of the fat particles as a pretreatment before anaerobic digestion. In all the aerobic and anaerobic digestion of solid waste, recirculation of the leachate is done with or without neutralization.

CN 1460423 the microorganisms used are Bacillus, photosynthetic bacterium, microzyme, Aspergillus oryzae and basidiomycetes. In this patent waste straw and stalk materials are inoculated with the above said microbes to undergo solid fermentation. Since they have used photosynthetic bacterium as one of the seed components the work was carried out in the presence of sunlight and open to atmosphere. The process of the present invention is carried out under anaerobic condition in the absence of open atmosphere and sunlight.

U.S. Pat. No. 3,711,392 discloses mechanically pulverized aqueous suspension of biodegradable waste materials like cellulose, sulfur or sulfur compounds and hydrocarbon oil in the fermentation tank, which are supplemented with derivatives of acetic acid, carbon tetra chloride, steering agents, emulsifying agents and one or more antibiotics. This was mixed with microbes of various types like cellylolytic microorganisms, protozoa, cheese curing bacteria, molds, hydrogen bacteria, yeast, sulfur bacteria and antibiotic resistant inoculums. Solid proteinous materials are separated at the isoelectic point of the protein by autolyzing at high voltage alternating potential and coagulating the protein and processing into food. Other compounds are separated into various compounds using electrodialysis and ion selective membranes. Nutrients were added and liquor is recirculated. In the method of the present invention no separation of various compounds is done. All the constituents of the solid organic matter are liquefied at a pH of not less than 3 under anaerobic condition for a period of not less than 5 days. No external chemicals/nutrients are added. Liquefaction takes place without the aid of mechanical equipments.

CN1500862 relates to one kind of microbial solid fermenting agent and its preparing process to produce organic fertilizer, protein feed etc. in this process cultured microbial seed are used for treating waste. Nutrients are also added. In the process of the present invention, no cultured microbial seeds are used. The fermentative microbes like bacteriodes, Clostridium, Lactobacillus, Streptococcus, Peptococcus, Selenomonas in any combination present in the water are used for liquefaction.

JP2002102896 deals with improving the effective separation and dehydration properties of organic sludge. This method uses biological, physiochemical and mechanical liquefaction process. Sludge is re-circulated. In the present process, only biological liquefaction of solid organic mater is done. Sludge recirculation is not required.

JP7002589 discloses a fermentation product having high water retainability and water permeability and containing a slow acting fertilizing component by using microorganisms which can survive in alkaline environment in the presence of highly active calcium oxide having a pH of 12. In the present process, the biological liquefaction of organic matter takes place from pH of not less than 3.

JP2004042008 deals with garbage treatment device capable of simultaneously treating the wastewater produced by subjecting the garbage to fermentation. Microbial culture is introduced. Produced Carbon dioxide with moisture is removed through a degassing pipe using pump and the mixture is cooled by cooling bath to separate water. The CO2 is again returned to the fermenter and the water is treated by aeration. Whereas, in the claimed invention only the solid matter is converted into liquid and no recirculation of gas or water is involved. This is a liquefaction process.

In the process of CN1511940 the fermentation of dewatered animal feces was carried out using primary seed culture and secondary proliferation culture of Bacillus, non spore bearing Bacillus, Saccharomyces, Actinomycetes, photosynthetic bacterium, mycelial fungus and Lactobacillus and mixed with the solid in certain proportion. Supplementary materials also added to produce active microbial fermented product. However, in the process of the present invention no cultured microbial seeds are used. The fermentative microbes like Bacteriodes, Clostridium, Lactobacillus, Streptococcus, Peptococcus, Selenomonas in any combination present in the water are used for liquefaction of solid organic matter. No supplementary materials are required.

A detailed comparison of the conventionally known septic tank systems with the process of the present invention is tabulated in table 1 here as under:

TABLE 1

| S. No | Working of septic tank | Process of present invention |
|---|---|---|
| 1. | Septic tank is a digester | The claim is for liquefaction of |
| | | solids |
| 2. | Solid to liquid ratio in | Solid to liquid ratio is 1:1 to 3. |
| | sewage is about 0.1:99.9. | solid content in this process is |
| | solid content in | varying from 30 to 50% |
| | sewage is only 0.1% | |
| 3. | Septic tank acts like a | Minimum of 5 days is required to |
| | sedimentation tank with | convert the solid organic matter |
| | a provision for partial | to liquid. After liquefaction, |
| | sludge digestion where | the liquid can be treated in |
| | sewage is held for 1 to | conventional effluent treatment |
| | 2 days and the suspended | plants. |
| | solids settles at the | |
| | bottom and the gradual | |
| | decomposition will be | |
| | taking place. Generally | |
| | the sludge is allowed to | |
| | remain there for a | |
| | minimum of 12 months. | |
| 4. | Only About 30% of settled | During the liquefaction process |
| | solids are digested | more than 90% of complex solid |
| | anaerobically in the | organic matter is converted |
| | septic tank to gaseous | into soluble simpler organic |
| | end products like | compounds. |
| | carbon di oxide, methane | |
| | and hydrogen sulfide. | |
| 5. | Flow to septic tank is | Liquefaction is done in batch |
| | intermittent. | operation with fixed solid to |
| | | liquid ratio composition |
| 6. | Suitable for only low | This can be applicable for high |
| | solid content wastewater | solid content |
| 7. | This is applicable for | All types of degradable organic |
| | uniform settleable | solid matter such as fleshing, |
| | solids only | vegetable wastes, chicken |
| | | intestine etc can be liquefied |
| | | in this process |

In the Thesis entitled “Anaerobic digestion of Biodegradable organics in municipal waste” by Shefali Verma the key features include pretreatment or sorting and shredding of the organics, heat exchanger to maintain the reactor temperature, pH control by addition of lime. Whereas, in the present invention no pretreatment or sorting or shredding is done. Heat exchanger not needed. No lime addition for pH correction is required.

In U.S. Pat. No. 6,013,183 treatment with alkali followed by liquefaction of microorganisms at high pressure of 10000-20000 psi is done. Whereas, in the present patent no chemical addition or liquefaction of microorganisms at high pressure is carried out. The study is conducted at atmospheric pressure.

Thus, keeping in view all that has been reported in the literature the inventors realized that there exists a dire need to provide a simple eco-friendly biological method to utilize the solid organic wastes for useful purposes.

None of the microorganisms used in the present invention are reported for converting solid organics into liquid in the conventional processes. In the present patent, these microorganisms are used only for conversion of solid organics into liquid. Minimum microbial population density of 10²/ml is needed.

The novelty and non obviousness of the present invention lies in the biological liquefaction of solid organic matter into liquid using anaerobically treated effluent under anaerobic condition to sugar, fatty acids, amino acids, alcohols without pretreatment like shredding of the solid organics, addition of chemicals, enzymes, recirculation of water and maintaining the reactor temperature with heat exchanger. The novelty of the present invention also resides in the treatment of the solid organic matter with fermentative microorganisms as herein described, under anaerobic condition to convert the same into liquid, thereby providing a simple eco-friendly biological method to utilize the solid organic wastes for useful purpose.

### BRIEF SUMMARY OF THE INVENTION

The present invention provides a novel process for liquefaction of solid organic matter, which obviates the drawbacks as, detailed above.

The present invention also provides a novel microbial consortium [MTCC 5186] for the liquefaction of solid organic matter.

Still another embodiment of the present invention provides a simple and economically viable method to liquefy the solid organic matter so that it can be treated in existing effluent treatment systems.

Yet another embodiment of the present invention provides a process that requires less hydraulic retention time, decreases the total space and time requirement as compared to conventional methods.

A further embodiment of the present invention provides a simple biological process that does not need any equipment like mincer and homogenizer for pulverizing the solid organic matter thereby reducing the costs towards electricity.

Another embodiment of the present invention provides a process in which the Hydraulic Retention Time is reduced to a minimum of 35% as the solid organic matter is hydrolyzed into simpler organic acid during liquefaction.

Yet another embodiment of the present invention provides a process that does not require any temperature adjustments since liquefaction is done at ambient temperature of about 25° C. to 40° C.

Still another embodiment of the present invention provides a process that overcomes the problems encountered in conventional methods like scum formation, floating of feed material leading to incomplete digestion, blocking of inlet and outlet pipes.

Accordingly, the present invention provides a novel microbial consortium comprising the bacteria of the genera Bacteroides, Clostridium, Lactobacillus, Streptococcus, Peptococcus, Selenomonas in any combination for the liquefaction of solid organic matter.

The present invention also provides a novel process for the liquefaction of solid organic matter, which comprises treating solid organic matter with 100% to 300% w/v, of water containing fermentative microorganisms with a minimum population density of 10² per ml under anaerobic conditions at a pH of not less than 3.0 for a period of minimum 5 days, preferably with intermittent stirring, at a temperature in the range of 25 to 40 degree C. to obtain the liquefied organic matter

In an embodiment of the present invention the fermentative microorganism used for liquefaction may be selected from Bacteroides, Clostridium, Lactobacillus, Streptococcus, Peptococcus, Selenomonas in any combination.

In another embodiment of the present invention the solid organic matter is treated with 100% to 300% w/v of water containing the said consortium with a minimum population density of 10² per ml under anaerobic conditions.

In a further embodiment of the present invention the solid organic matter is preferably domestic and industrial organic solid matter generated from tanning industries, poultry farms, food processing industries, sago and starch industries, agriculture, slaughter houses, meat and fish stalls, chicken broiler stalls, Vegetable and fruit markets, restaurants, hostels, residences and commercial complexes.

In yet another embodiment of the present invention the population density of the microbial consortium is preferably 10³-10⁴ per ml.

In another embodiment of the present invention the liquefaction is carried out at pH preferably 10-12.

In a further embodiment of the present invention the process can be coupled to any existing waste treatment system preferably septic tank system.

### DETAILED DESCRIPTION OF THE INVENTION

The microbial consortium was taken out from the treated wastewater effluent coming out of the pilot scale Upflow Anaerobic Sludge Blanket (UASB) reactor treating tannery wastewater. The 12.5 m³ capacity UASB reactor is in the premises of CLRI, Chennai. Feed for the UASB reactor comprises of the composite wastewater from tanneries which contains proteins, fats, keratin etc. The effluent from the UASB reactor harbors the microorganisms utilized for the purposes of the present invention. The ratio of the microorganisms in the wastewater varies from batch to batch depending upon the characteristics of the influent feed. However, the said ratio does not affect the quality of the treatment process in any way and is thus not reflected in the specification. The major characteristics of the microbes used for the present invention are presented below in table 2. The microorganisms used for the purpose of the present invention are deposited as a consortium under Accession No. MTCC 5186.

TABLE 2

| | Name of the | |
|---|---|---|
| S. No | microorganism | Characteristics of the microorganisms |
| 1. | Bacteroides | Facultatively anaerobic, Gram negative, rods |
| | | of short to moderately long, non spore |
| | | forming, degrade bio macro molecules like |
| | | gelatin, starch, cellulose, pectin |
| 2. | Clostridium | Anaerobic, gram positive bacteria, rod shaped. |
| | | Gelatin, carbohydrates such as starch, |
| | | cellulose, zylans and lactose can be |
| | | fermented into volatile fatty acids and |
| | | alcohols |
| 3. | Lactobacillus | Anaerobic, gram positive bacteria, rod |
| | | shaped, non spore forming. carbohydrates, |
| | | amino acids, fatty acid esters ferment |
| | | into acids. |
| 4. | Streptococcus | Anaerobic, gram-positive bacteria. They |
| | | hydrolyze a wide range of macromolecules |
| | | including cellulose, chitin, proteins |
| 5. | Peptococcus | Anaerobic, gram-positive bacteria, cocci |
| | | shaped help in carbohydrates and proteins |
| | | fermentation |
| 6. | Selenomonas | Anaerobic, gram negative, curved or crescent |
| | | shaped rods. In general selenomonas are |
| | | obligately saccharolytic, although some |
| | | strains ferment lactate or amino acids. |

Solid organic matter is treated with 100% to 300% of water containing fermentative microorganisms at a pH of not less than 3.0 under anaerobic condition for a period of minimum 5 days preferably with intermittent stirring at a temperature in the range of 25 to 40° C. The complex organic matter is hydrolyzed into simpler compounds by the fermentative microorganisms to produce liquefied organic matter. Liquefied organic matter is defined as soluble polymers or monomers with low molecular weight which can cross the cell barrier. Liquefied organic matter comprises of soluble simpler compounds like sugar, fatty acids, amino acids, alcohol etc.

The following examples are given by way of illustration and therefore should not be construed to limit the scope of the present invention.

### EXAMPLE—1

In a 120 L capacity container 50 kg of tannery limed fleshing from hide was treated with 50 L of effluent from anaerobic reactor containing the microbial population density of 10³ per mL with the fermentative microorganisms of Bacteroides, Clostridium, Lactobacillus, Streptococcus, Peptococcus, Selenomonas, The pH of the mixture was 12.0. The contents were kept in a closed container for anaerobic condition. Stirring was given intermittently for 10 minutes at 15 rpm for every 6 hours. The time taken for liquefaction was 12 days. Then it was filtered through 5 mm sieve and it was observed that 80% of limed fleshing was liquefied. COD in the liquefied limed fleshing was 75 g/L.

The liquefied material can be directly used for biogas production by digesting them in an anaerobic reactor. The quantity of biogas produced was measured using gas flow meter. 55 Liters of biogas was produced per kg of wet fleshing with 85% moisture generated from the hide.

### EXAMPLE—2

In a 130 L capacity container 4 kg of tannery limed fleshing from hide was treated with 80 L of anaerobically treated water containing the microbial population density of 10⁴ per mL with the fermentative microorganisms of Bacteroides, Clostridium, Lactobacillus, Streptococcus, Peptococcus, Selenomonas. The pH value of the mixture was about 11.0. The contents were kept in a closed container for anaerobic condition. Stirring was given intermittently for 10 minutes at 15 rpm for every 6 hours. The time taken for liquefaction was 10 days. Then it was filtered through 5 mm sieve and it was observed that 90% of limed fleshing was liquefied. COD in the liquefied limed fleshing was 55 g/L.

The liquefied material can be directly used for biogas production by digesting them in an anaerobic reactor. The quantity of biogas produced was measured using gas flow meter. 58 liters of biogas was produced per kg of wet limed fleshing with 85% moisture generated from the hide.

### EXAMPLE—3

In a 130 L capacity container 40 kg of limed tannery fleshing from skin was treated with 80 L of water containing the microbial population density of 10³ per mL with the fermentative microorganisms of Bacteroides, Clostridium, Lactobacillus, Streptococcus, Peptococcus, Selenomonas. pH of the mixture was about 11.0. The contents were kept in a closed container for anaerobic condition. Stirring was given intermittently for 10 minutes at 15 rpm for every 6 hours. The time taken for liquefaction was 8 days. Then it was filtered through 5 mm sieve and it was observed that 90% of limed fleshing was liquefied. COD of the liquefied limed fleshing was 58 g/L.

The liquefied material can be directly used for biogas production by digesting them in an anaerobic reactor. The quantity of biogas produced was measured using gas flow meter. 60 L of biogas was produced per kg of wet limed fleshing with 85% moisture generated from the skin.

### EXAMPLE—4

In a 2 L capacity container 500 g of limed fleshing from skin was treated with 1.5 L of water containing the microbial population density of 10² per mL with the fermentative microorganism of Bacteroides, Clostridium, Lactobacillus, Streptococcus, Peptococcus, Selenomonas. The temperature was controlled at 30° C. pH of the mixture was about 10.0. Stirring was given intermittently for 60 seconds at 50 rpm for every 12 minutes. The time taken for liquefaction was 6 days. Then it was filtered through 5 mm sieve and it was observed that 90% of limed fleshing was liquefied. COD in the liquefied limed fleshing was 38 g/L.

The liquefied material can be directly used for biogas production by digesting them in an anaerobic reactor. The quantity of biogas produced was measured using gas flow meter. 59 liters of biogas was produced per kg of wet limed fleshing with 85% moisture generated from the skin.

### EXAMPLE—5

In a 5 L capacity container 500 g of chicken intestinal waste was treated with 1.5 L of anaerobically treated water containing the microbial population density of 10² per mL with the fermentative microorganisms of Bacteroides, Clostridium, Lactobacillus, Streptococcus, Peptococcus, Selenomonas. The experiment was done at the controlled temperature of 30° C. pH of the mixture was about 6.30. Stirring was given intermittently for 60 seconds at 50 rpm for every 12 minutes. The time taken for liquefaction was 12 days. Then it was filtered through 5 mm sieve and it was observed that 88% of limed fleshing was liquefied. COD of liquefied chicken intestinal waste was 62 g/L.

The liquefied material can be directly used for biogas production by digesting them in an anaerobic reactor. The quantity of biogas produced was measured using gas flow meter. 66 liters of biogas was produced per kg of wet chicken intestinal waste with 73% moisture content.

### EXAMPLE—6

In a 5 L capacity container 1000 g of chicken intestinal waste was treated with 2.0 L of water containing the microbial population density of 10² per mL with the fermentative microorganisms of Bacteroides, Clostridium, Lactobacillus, Streptococcus, Peptococcus, Selenomonas. The temperature was controlled at 30° C. pH of the mixture was about 3.50. Stirring was given intermittently for 60 seconds at 50 rpm for every 12 minutes. The time taken for liquefaction was 13 days. Then it was filtered through 5 mm sieve and it was observed that 93% of limed fleshing was liquefied. COD of the liquefied chicken intestinal waste was 132 g/L.

The liquefied material can be directly used for biogas production by digesting them in an anaerobic reactor. The quantity of biogas produced was measured using gas flow meter. 60 liters of biogas was produced per kg of wet chicken intestinal waste with 73% moisture content.

### EXAMPLE—7

In a 5 L capacity container 1000 g of Vegetable market waste was treated with 2.5 L of water containing the microbial population density of 10³ per mL with the fermentative microorganisms of Bacteroides, Clostridium, Lactobacillus, Streptococcus, Peptococcus and Selenomonas. The temperature was controlled at 30° C. pH of the mixture was about 6.50. Stirring was given intermittently for 60 seconds at 50 rpm for every 12 minutes. The time taken for liquefaction was 5 days. Then it was filtered through 5 mm sieve and it was observed that 90% of limed fleshing was liquefied. COD of the liquefied vegetable market waste was 45 g/L.

The liquefied material can be directly used for biogas production by digesting them in an anaerobic reactor. The quantity of biogas produced was measured using gas flow meter. 70 liters of biogas was produced per kg of vegetable market waste.

Advantages of the Present Invention Include the Following:

1. This process is a simple method and economically viable to liquefy the solid organic matter so that it can be treated in existing effluent treatment systems.

2. The ground water is protected from pollution arising due to indiscriminate dumping of solid organic matter in the open land after adopting this liquefaction process.

3. This technology can be easily adopted for domestic and industrial organic solid matter generated from tanning industries, poultry farms, food processing industries, sago and starch industries, agriculture, slaughter houses, meat and fish stalls, chicken broiler stalls, Vegetable and fruit markets, restaurants, hostels, residences and commercial complexes.

4. Less hydraulic retention time decreases the total space and time requirement as compared to conventional methods.

5. This process helps in elimination of air pollution due to putrefaction of dumped solid organic matter in open land.

6. This is a simple biological process and it does not need any equipment like mincer and homogenizer for pulverizing the solid organic matter thereby cost towards electricity will be greatly saved.

7. Liquefaction process is speeded up with anaerobically treated effluents that contain fermentative microorganisms.

8. This process ensures that toxicity to methanogens due to ammonia in anaerobic treatment unit is avoided since fleshing generated after liming process is in high pH so that free ammonia escapes during liquefaction thereby ensuring further process for biogas production.

9. By this process Hydraulic Retention Time is reduced to a minimum of 35% as the solid organic matter is hydrolyzed into simpler organic acid during liquefaction.

10. The process is not required any temperature adjustment conditions since liquefaction is done at ambient temperature of about 28° C. to 40° C.

11. The liquefied solid organic matter obtained after the process produces biogas that generates electrical energy.

12. By this process problems like scum formation, floating of feed material leading to incomplete digestion, blocking of inlet and outlet pipes are completely avoided that encounter in conventional methods.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A novel microbial consortium MTCC 5186 comprising bacteria of the genera Bacteroides, Clostridium, Lactobacillus, Streptococcus, Peptococcus, Selenomonas in any combination for the liquefaction of solid organic matter.

2. A consortium as claimed in claim 1, wherein the solid organic matter is treated with 100% to 300% w/v of water containing the said consortium with a minimum population density of 10² per ml under anaerobic conditions.

3. A novel process for liquefaction of solid organic matter, which comprises treating solid organic matter with 100% to 300% w/v, of water containing fermentative microorganisms with a minimum population density of 10² per ml under anaerobic conditions at a pH of not less than 3.0 for a period of minimum 5 days, preferably with intermittent stirring, at a temperature in the range of 25 to 40 degree C. to obtain the liquefied organic matter.

4. A process as claimed in claim 3, wherein the solid organic matter comprises domestic and industrial organic solid matter generated from one or more of a tanning industry, a poultry farm, a food processing industry, a sago and starch industry, agriculture, a slaughter house, a meat and fish stall, a chicken broiler stall, a vegetable or fruit market, a restaurant, a hostel, a residence and a commercial complex.

5. A process as claimed in claim 3, wherein the fermentative microorganism used for liquefaction is selected from bacteria of the genera Bacteroides, Clostridium, Lactobacillus, Streptococcus, Peptococcus, and Selenomonas in any combination.

6. A process as claimed in claim 3, wherein the population density of the microbial consortium is preferably 10³-10⁴ per ml.

7. A process as claimed in claim 3, wherein the liquefaction is carried out at pH preferably 10-12.

8. A process as claimed in claim 3, wherein the liquefaction is carried out at temperature preferably 30° C.-35° C.

9. A process as claimed in claim 3, wherein the process can be coupled to any existing waste treatment system preferably septic tank system.

10. A novel microbial consortium MTCC 5186 substantially as herein described with reference to the foregoing examples.
